(19) United States
(12) Patent Application Publication
(10) Pub. No.: US 2026/0086074 A1
(43) Pub. Date: Mar. 26, 2026

(54) PHOTOLITHOGRAPHIC FABRICATION OF SILICON PILLAR ARRAYS WITH PERFORATED TOP ELECTRODE FOR TRACE VAPOR

(71) Applicant: The Government of the United States of America, as represented by the Secretary of the Navy, Arlington, VA (US)

(72) Inventors: Daniel Ratchford, Washigton, DC (US); Braden Giordano, Washigton, DC (US); Junghoon Yeom, Washigton, DC (US)

(73) Assignee: The Government of the United States of America, as represented by the Secretary of the Navy, Arlington, VA (US)

(21) Appl. No.: 19/336,981

(22) Filed: Sep. 23, 2025

Related U.S. Application Data
(60) Provisional application No. 63/698,122, filed on Sep. 24, 2024.

(51) Int. Cl. G01N 33/00; B82Y 15/00; G01N 1/22; G01N 1/40; G03F 7/00

(52) U.S. Cl. CPC G01N 33/0019; G01N 1/2214; G01N 1/405; G03F 7/0035; B82Y 15/00

(57) ABSTRACT

Systems and methods are provided for improving the fabrication of silicon pillar arrays and porous top electrodes for trace vapor preconcentration and partial separation. In an embodiment, the silicon pillar arrays are fabricated using photolithography or maskless photolithography combined with a dry etching process. Importantly, this process is more reproducible and scalable than the past fabrication method and yields better device performance.

### CROSS REFERENCE TO RELATED APPLICATIONS

This application claims the benefit of U.S. Provisional Patent Application Number 63/698,122, filed on Sep. 24, 2024, which is incorporated by reference herein in its entirety.

### FEDERALLY SPONSORED RESEARCH AND DEVELOPMENT

The United States Government has ownership rights in this invention. Licensing inquiries may be directed to Office of Technology Transfer at US Naval Research Laboratory, Code 1004, Washington, DC 20375, USA; +1.202.767.7230; nrltechtran@us.navy.mil, referencing Navy Case Number 212343-US2.

### FIELD OF THE DISCLOSURE

This disclosure relates to lithography, including photolithography.

### BACKGROUND

The sensing of trace explosives in complex environments is a critical need and significant challenge for the Navy. Vertical pillar arrays can be used for enhancement of trace chemical sensing through preconcentrate and partial separation of trace vapors. The pillar arrays can serve as high surface area adsorptive substrates for trace vapor adsorption in a noncontact/standoff mode of operation. They can enable rapid and controlled Joule heating profiles that provide unique thermal desorption spectra for component analysis by portable multichannel detectors (i.e. mass spectrometer or ion mobility spectrometer).

The structure of the preconcentration device can include a vertically aligned array of silicon pillars attached at their base to a silicon wafer. The tops of the silicon pillars can be coated with a perforated metal electrode, which allows for electrical contact to be made to the tops of the pillars while allowing molecules to pass through the electrode and absorb to the pillar sidewalls. The central portion of each silicon pillar is highly resistive, such that a bias applied between the top electrode and the silicon substrate results in Joule heating of the pillars.

Conventional fabrication methods used to create preconcentration devices have several issues that can limit device performance. The conventional fabrication process used a combination of nanosphere lithography and metal-assisted chemical etching to form vertically aligned silicon nanopillars. The process started with forming a close-packed monolayer of polystyrene nanospheres with submicron diameters on top of a silicon substrate. Then, the nanospheres were etched with an oxygen plasma to reduce their diameters. A gold film was evaporated on top of the nanospheres and the nanospheres were removed. A perforated gold film was left behind on top the silicon wafer with holes the size of the reduced nanosphere diameter and a pitch between the holes determined by the original nanosphere diameter. Metal-assisted chemical etching was used to form the silicon nanopillars by immersing the wafer in a solution of HF and H₂O₂, where gold selectively and anisotropically etched into the silicon substrate, leaving behind a well-ordered array of vertically standing nanopillars. Next, another nanosphere lithography step was used to form the porous top electrode. The nanopillar array was backfilled with photoresist, a monolayer of nanospheres was deposited on top the nanopillars, and the nanospheres were etched to reduce their diameters. A metal film was then evaporated on top of the nanospheres, and the nanospheres were then removed leaving behind a porous top electrode on top of the nanopillars.

This fabrication process has several disadvantages. First, the formation of a nanosphere monolayer relies on a self-assembly mechanism that often produces micron-to millimeter-scale voids (“hole regions) in the monolayer. These hole regions create large gaps in the silicon array where no nanopillars are present, as well as corresponding large pore-free areas in the porous top electrode, thereby reducing the effective active area of the device. Second, the nanospheres frequently stack into multiple layers during self-assembly (i.e., multiple stacked regions). These multiple stacked regions prevent uniform etching of the underlying silicon during the subsequent metal-assisted chemical etching step, leaving large unetched areas that can create electrical shorts. In the case of the porous top electrodes, the stacked nanosphere regions create oversized holes that further reduce the effective active area of the device. Because the distribution hole and multilayer regions in the nanosphere film is inherently random, the silicon nanopillar array experiences nonuniform etch rates. This inconsistency arises from the strong dependence of the metal-assisted chemical etching process on the amount of local gold coverage. For the porous top electrode, the removal of the nanospheres is challenging and frequently the nanospheres cannot be completely removed, further reducing the active device area. Taken together, the stochastic nature of nanosphere monolayer formation results in variations in nanopillar surface density, the presence of electrical shorts, and a nonuniform porous top electrode, all of which contribute to a highly variable device performance.

### BRIEF DESCRIPTION OF THE DRAWINGS/FIGURES

The accompanying drawings, which are incorporated in and constitute part of the specification, illustrate embodiments of the disclosure and, together with the general description given above and the detailed descriptions of embodiments given below, serve to explain the principles of the present disclosure. In the drawings:

FIG. 1 is a diagram showing parts of a silicon wafer used to form the silicon pillar preconcentrators in accordance with an embodiment of the present disclosure;

FIG. 2 shows optical images of photoresist disks on top of a silicon substrate in accordance with an embodiment of the present disclosure;

FIG. 3A shows Scanning Electron Microscope (SEM) images of the silicon pillars with diameters of ˜3 μm and heights of ˜9 μm in accordance with an embodiment of the present disclosure;

FIG. 3B shows an illustration of a side view of silicon pillars in accordance with an embodiment of the present disclosure.

FIG. 4 shows an illustration depicting an exemplary fabrication process in accordance with an embodiment of the present disclosure;

FIG. 5 shows an optical image, a SEM image of silicon pillars, and a SEM top down image of the silicon pillar preconcentrator after top electrode deposition in accordance with an embodiment of the present disclosure; and

FIG. 6 is a diagram showing a 5 times increase in detection sensitivity in accordance with an embodiment of the present disclosure.

Features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

In the following description, numerous specific details are set forth to provide a thorough understanding of the disclosure. However, it will be apparent to those skilled in the art that the disclosure, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring aspects of the disclosure.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to understand that such description(s) can affect such feature, structure, or characteristic in connection with other embodiments whether or not explicitly described.

### 1. Overview

Embodiments of the present disclosure improve the fabrication of silicon pillar arrays and porous top electrodes for trace vapor preconcentration and partial separation. In an embodiment, the silicon pillar arrays are fabricated using photolithography or maskless photolithography combined with a dry etching process. Importantly, this process is more reproducible and scalable than the past fabrication method and yields better device performance.

### 2. Exemplary Process

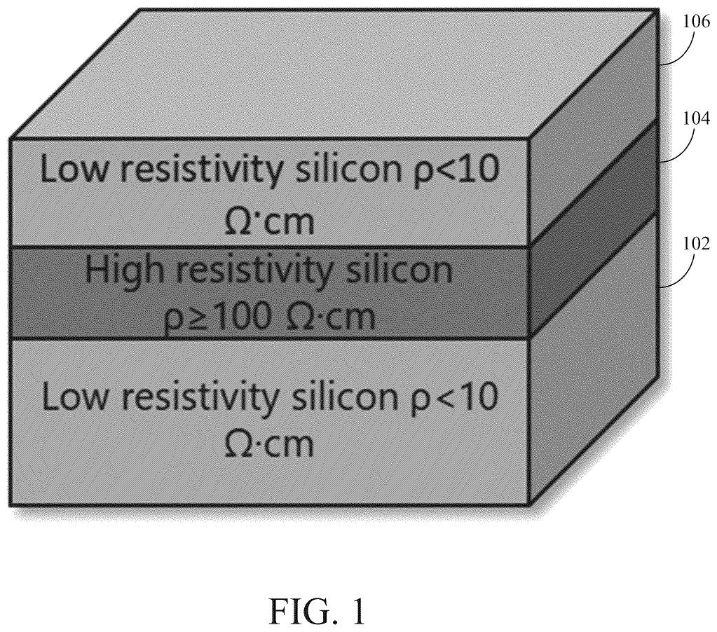

In an embodiment, vertically aligned silicon pillar arrays are formed using a combination of photolithography and a dry etch process. In an embodiment, silicon pillar preconcentrators are made from a silicon wafer that has two epitaxial grown layers of silicon with varying resistivity on top of it (see FIG. 1). In an embodiment, the substrate and top epitaxial layer have low resistivities (resistivity <10 Ω·cm), and a high resistivity intrinsic silicon layer (resistivity >100 Ω·cm) is placed in between these layers. In an embodiment, the thicknesses of the top and intrinsic layers are a few microns thick each.

FIG. 1 is a diagram showing parts of a silicon wafer used to form the silicon pillar preconcentrators in accordance with an embodiment of the present disclosure. FIG. 1 shows a low resistivity substrate, 102, a high resistivity intrinsic silicon layer 104, and a low resistivity top epitaxial layer 106.

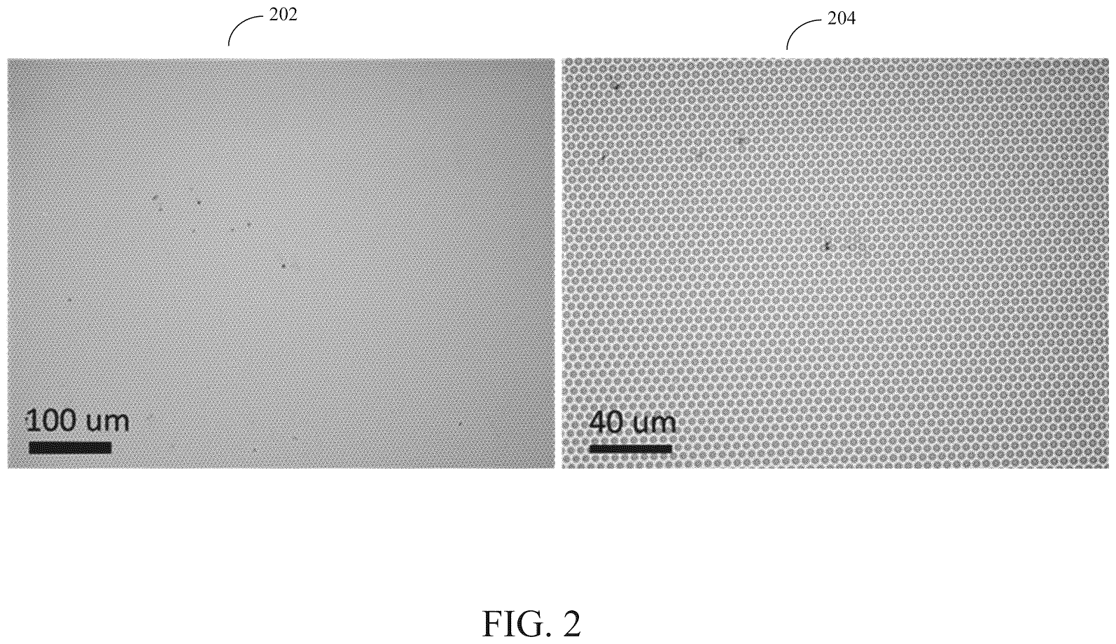

In an embodiment, to fabricate the silicon pillars, the silicon wafer is coated with photoresist, and either standard photolithography or direct write lithography is used to pattern the photoresist with arrays of disks (see FIG. 2). In an embodiment, the disks can be arranged in a hexagonal close-packing or square array pattern, and the disks have diameters and gaps between the pillars ranging from 100s of nanometers to a few microns. Using the photoresist disks as etch masks, the silicon can be etched with a mixture of SF₆ and O₂ gases. In an embodiment, the silicon is etched past the high resistivity to region so that the high resistivity region is in the core of the pillars.

FIG. 2 shows optical images of photoresist disks on top of a silicon substrate in accordance with an embodiment of the present disclosure. FIG. 2 shows image 202 with a 100 μm scale bar and image 204 with a 40 μm scale bar. In this example, direct write lithography with a negative photoresist (NR9-1000PY) was used to fabricate the disk arrays.

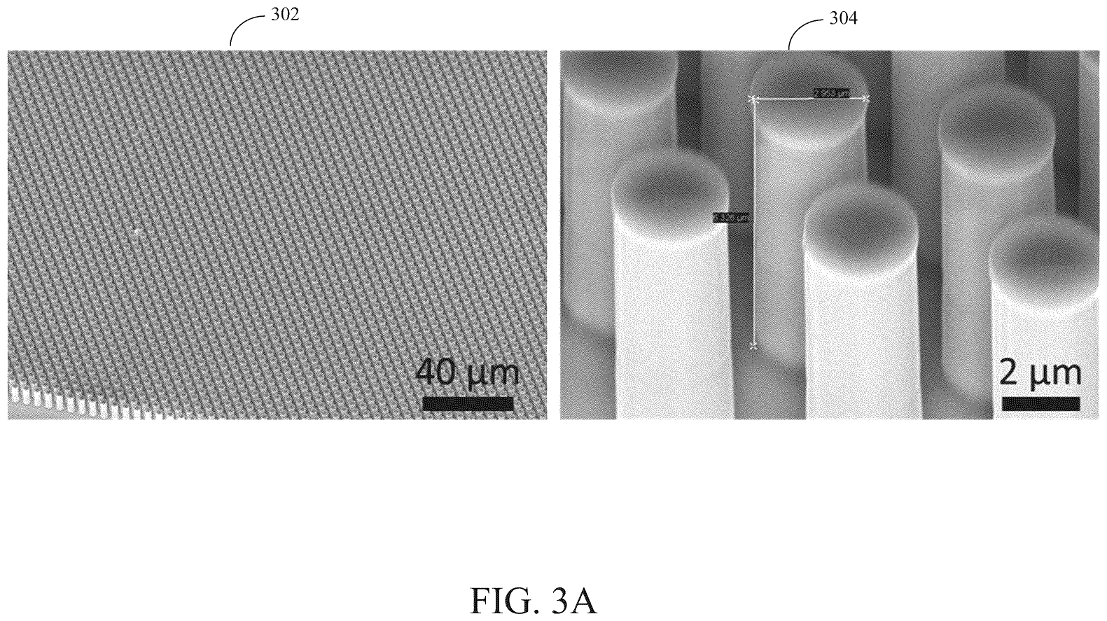

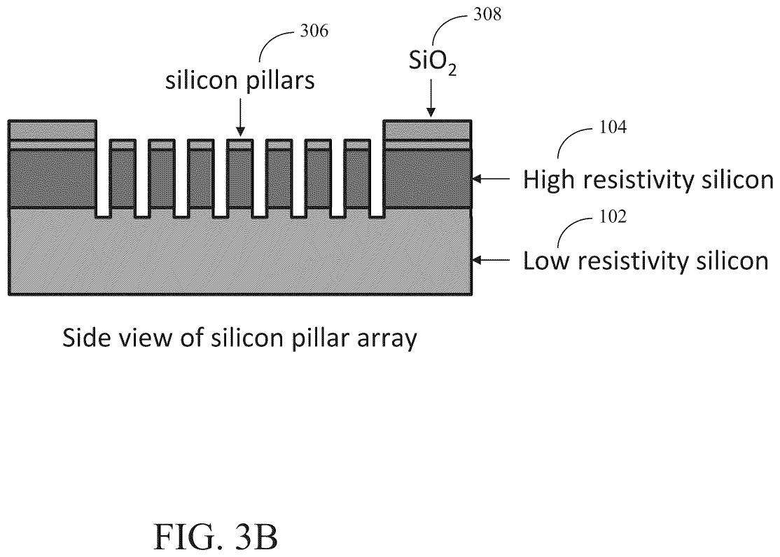

FIG. 3A shows Scanning Electron Microscope (SEM) images of the silicon pillars with diameters of ˜3 μm and heights of ˜9 μm in accordance with an embodiment of the present disclosure. FIG. 3A shows image 302 with a 40 μm scale bar and image 304 with a 2 μm scale bar. In FIG. 3A, etching for 5 minutes resulted in 9 μm long silicon pillars. The pillar arrays span millimeter scale regions and contains millions of pillars. The silicon arrays can be rinsed in an acetone bath to remove the photoresist from the tops of the silicon pillars.

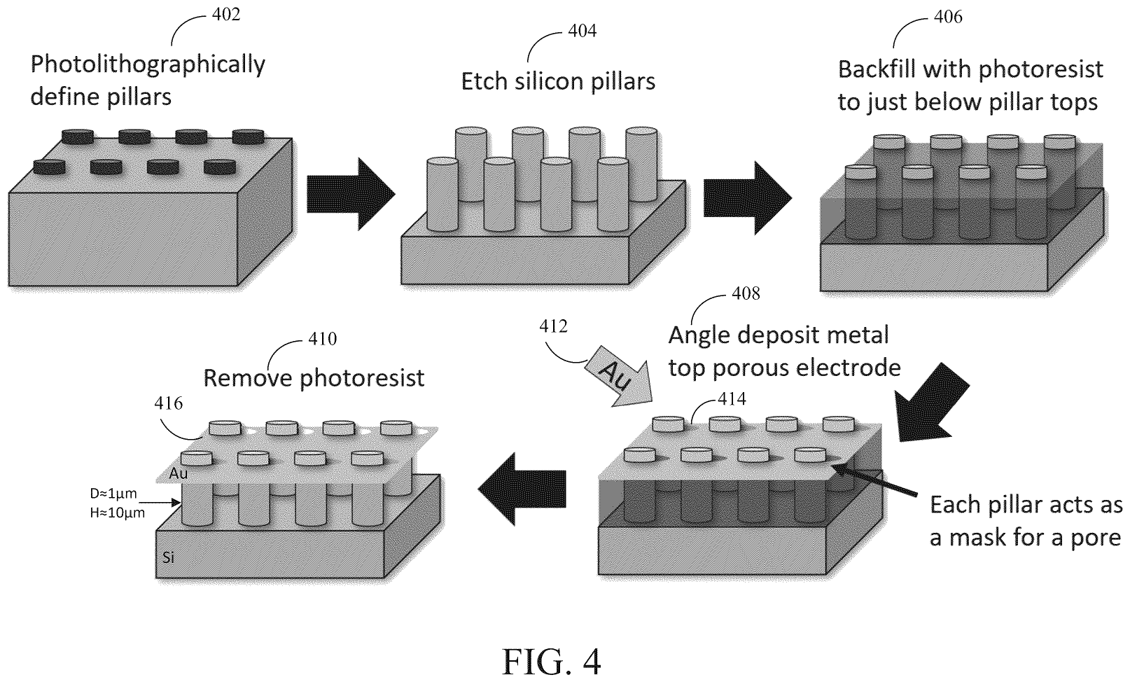

In an embodiment, to make electrical contact to the tops of the pillars, SiO₂ contact pads are deposited at the edge of the silicon pillar array. The SiO₂ pads isolate the top electrical connection from the substrate. In an embodiment, to make these pads, a SiO₂ film is deposited onto the silicon, and the SiO₂ is selectively etched from the silicon pillar region, leaving the SiO₂ surrounding the array. In an embodiment, top contact to the pillars is made using the process depicted in FIG. 4.

FIG. 3B shows an illustration of a side view of silicon pillars in accordance with an embodiment of the present disclosure. FIG. 3B shows silicon pillars 306 etched into low resistivity substrate 102 and high resistivity intrinsic silicon layer 104. FIG. 3B also shows SiO₂ film 308 deposited at the edges of the silicon pillar array 306.

FIG. 4 shows an illustration depicting an exemplary fabrication process in accordance with an embodiment of the present disclosure. In step 402, pillars are photolithographically defined. In step 404, silicon pillars are etched. In step 406, photoresist is used to backfill the negative space between the pillars to a height of about 0.1-2 μm below the pillar tops. In an embodiment, the silicon pillar array is coated with a thick layer of photoresist that is then etched back with an oxygen plasma to reveal the tops of the silicon pillars. In step 408, a metal (e.g., gold) is deposited on top of the photoresist at an angle. In an embodiment, each pillar acts as a mask for a pore, and pore size is guaranteed and controlled by the metal deposition angle. In step 410, photoresist is removed.

In an embodiment, to make the top electrode, gold 412 is deposited onto the array at a shallow angle such that the tops of the pillars act as shadow masks, creating a crescent shaped hole (e.g., hole 414) beside each pillar. In an embodiment, the size of the hole can be controlled with deposition angle. In an embodiment, acetone is used to remove the photoresist, resulting in a free-standing top electrode 416.

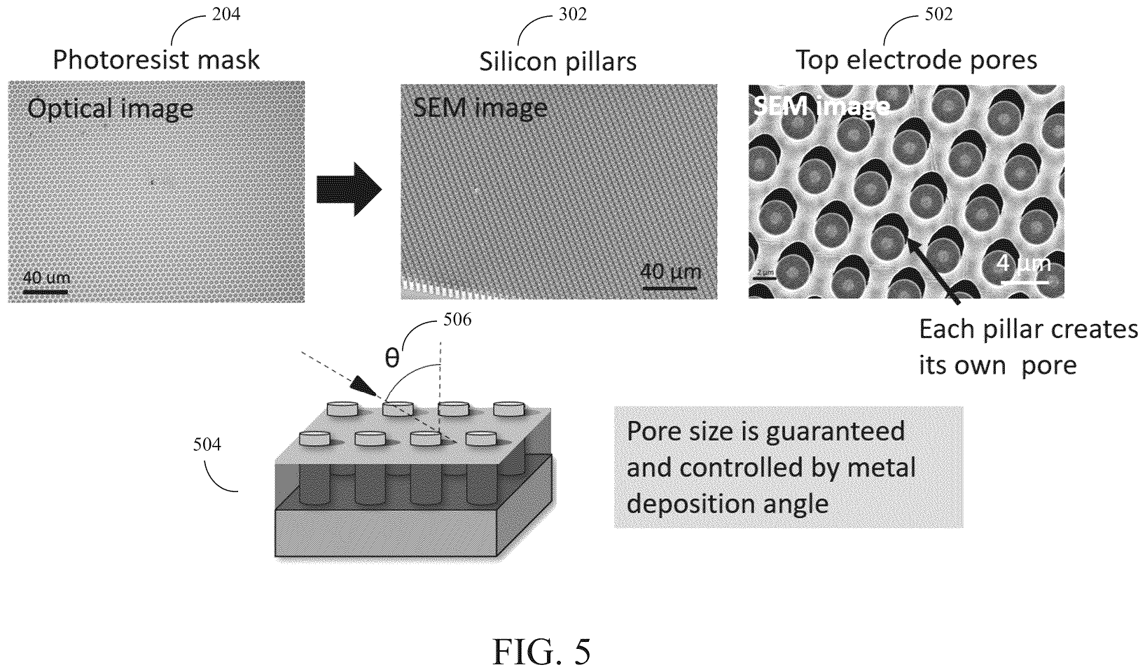

FIG. 5 shows an optical image, a SEM image of silicon pillars, and a SEM top down image 502 of the silicon pillar preconcentrator after top electrode deposition in accordance with an embodiment of the present disclosure. In FIG. 5, image 502 shows an SEM image of a silicon preconcentrator device after formation of the top electrode and shows the crescent shaped hole besides each pillar which allows gases to diffuse into the silicon pillar array. In an embodiment, a back ohmic contact to the array is made by depositing metal on the bottom of the samples. In an embodiment, any common metal electrode materials like silver (Ag), copper (Cu), platinum (Pt), and aluminum (Al) can be used here. In an embodiment, the silicon pillars are annealed to reduce the contact resistance between the pillars and top porous electrode.

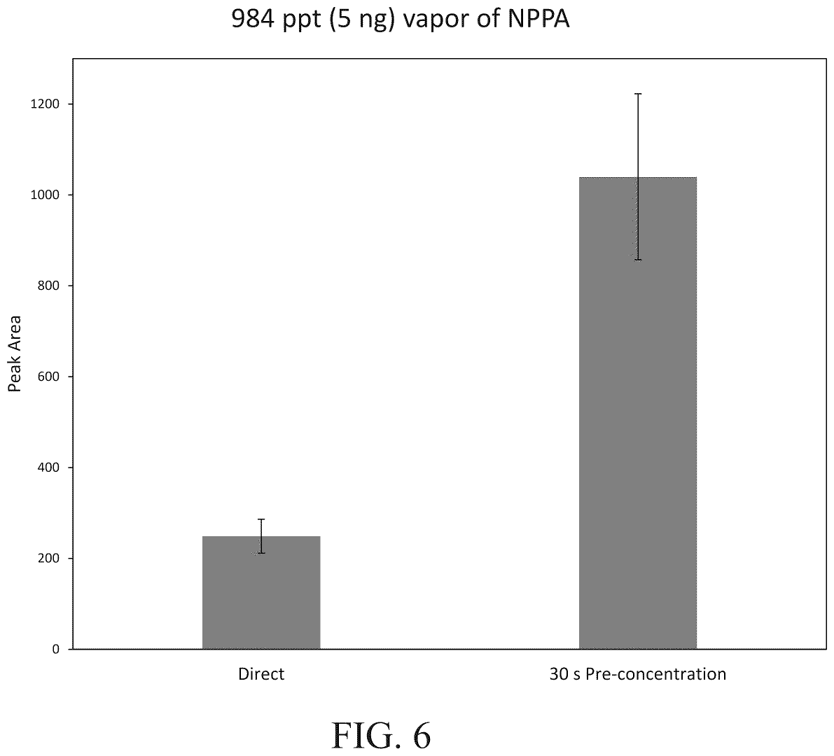

In an embodiment, the process shown in FIG. 4 has been used to make silicon pillar preconcentrators and has shown trace vapor preconcentration and separation. The photolithographic process of FIG. 4 yields reproducible, uniform pillars, a porous top electrode, and eliminates electrical shorts. Devices have been used for preconstruction of trace vapors. FIG. 6 is a diagram showing a 5-times increase in detection sensitivity in accordance with an embodiment of the present disclosure. Embodiments of the present disclosure provide improved performance of existing handheld trace vapor detection instrumentation through preconcentration.

### 3. Exemplary Advantages

Systems and methods in accordance with embodiments of the present disclosure result in pillar arrays and porous top electrodes with consistently uniform surface coverage. The overall impact is preconcentrators with less electrical shorts, better device performance, and improved device-to-device consistency compared to conventional fabrication processes. Advantages of embodiments of the present disclosure include: the uniformity and density of the silicon array and pore size are guaranteed; the process is highly reproducible and scalable; and the size of the pores can be controlled by a deposition angle.

### 4. Conclusion

It is to be appreciated that the Detailed Description, and not the Abstract, is intended to be used to interpret the claims. The Abstract may set forth one or more but not all exemplary embodiments of the present disclosure as contemplated by the inventor(s), and thus, is not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

While various embodiments of the present disclosure have been described above, it should be understood that they have been presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments.

What is claimed is:

1. A method for photolithographic fabrication of a silicon pillar array, the method comprising:
photolithographically defining a plurality of pillars on a silicon substrate;
etching the silicon pillars;
backfilling an area around the pillars with photoresist to a height just below the tops of the pillars;
depositing, at a predetermined angle, metal on top of the photoresist, wherein each pillar in the plurality of pillars acts as a mask for a respective pore in a plurality of pores when the metal is deposited at the predetermined angle, thereby creating the plurality of pores; and
removing the photoresist.

2. The method of claim 1, wherein photolithographically defining the pillars on the silicon substrate forms a plurality of silicon pillar preconcentrators.

3. The method of claim 1, wherein the plurality of pores are crescent-shaped holes positioned to the side of each pillar in the plurality of pillars.

4. The method of claim 1, wherein the pore size is guaranteed and controlled by the angle.

5. The method of claim 1, wherein the metal is gold.

6. The method of claim 1, wherein etching the silicon pillars comprises:
etching the silicon pillars with a mixture of SF₆ and O₂ gases using the photolithographically defined plurality of pillars as etch masks.

7. The method of claim 1, wherein the silicon substrate comprises a silicon wafer, and wherein the silicon wafer comprises a plurality of layers of silicon with varying resistivity.

8. The method of claim 7, wherein the plurality of layers comprise:
a low resistivity substrate;
a high resistivity intrinsic layer on top of the low resistivity substrate; and
a low resistivity top epitaxial layer on top of the high resistivity intrinsic layer.

9. The method of claim 8, wherein the low resistivity substrate and the low resistivity top epitaxial layer have resistivity <10 Ω·cm, and wherein the high resistivity intrinsic silicon layer has resistivity >100 Ω·cm.

10. The method of claim 8, wherein etching the silicon pillars comprises:
etching the silicon pillars past the high resistivity intrinsic layer such that the high resistivity intrinsic layer forms respective cores of respective pillars in the plurality of pillars.

11. The method of claim 1, wherein the plurality of pores are shaped such that gases can diffuse into the silicon pillar array.

12. The method of claim 1, further comprising:
depositing a metal onto the bottom of the silicon pillar array, thereby forming a back contact.

13. The method of claim 1, further comprising:
annealing the plurality of pillars such that contact resistance between the plurality of pillars and the metal is lowered.

14. The method of claim 1, wherein the metal forms an electrode.

15. A silicon pillar array, comprising:
a silicon substrate, comprising:
a low resistivity substrate, and
a high resistivity intrinsic layer on top of the low resistivity substrate;
a plurality of pillars extending upwards from the silicon substrate; and
a metal layer on top of the plurality of pillars, wherein the metal layer has a plurality of pores, and wherein each pore in the plurality of pores is positioned next to a respective pillar in the plurality of pillars.

16. The silicon pillar array of claim 15, wherein the metal is gold.

17. The silicon pillar array of claim 15, wherein the low resistivity substrate has resistivity <10 Ω·cm, and wherein the high resistivity intrinsic silicon layer has resistivity >100 Ω·cm.

18. The silicon pillar array of claim 15, wherein the plurality of pores are shaped such that gases can diffuse into the silicon pillar array.

19. The silicon pillar array of claim 15, wherein the metal forms an electrode.

20. A method for photolithographic fabrication of a silicon pillar array, the method comprising:
photolithographically defining a plurality of pillars on a silicon substrate, wherein the silicon substrate comprises:
a low resistivity substrate,
a high resistivity intrinsic layer on top of the low resistivity substrate, and
a low resistivity top epitaxial layer on top of the high resistivity intrinsic layer;
etching the silicon pillars past the high resistivity intrinsic layer such that the high resistivity intrinsic layer forms respective cores of respective pillars in the plurality of pillars;
backfilling an area around the pillars with photoresist to a height just below the tops of the pillars;
depositing, at a predetermined angle, metal on top of the photoresist, wherein each pillar in the plurality of pillars acts as a mask for a respective pore in a plurality of pores when the metal is deposited at the predetermined angle, thereby creating the plurality of pores; and
removing the photoresist.
